(19) United States
(12) Patent Application Publication
(10) Pub. No.: US 2021/0076946 A1
(43) Pub. Date: Mar. 18, 2021

(54) METHOD AND APPARATUS FOR DETECTING THE BODY TEMPERATURE OF A PATIENT

(71) Applicant: Fresenius Medical Care Deutschland GmbH, Bad Homburg (DE)

(72) Inventor: Reiner SPICKERMANN, Burghausen (DE)

(73) Assignee: Fresenius Medical Care Deutschland GmbH, Bad Homburg (DE)

(21) Appl. No.: 16/772,331

(22) PCT Filed: Dec. 14, 2018
(86) PCT No.: PCT/EP2018/085010

§ 371 Date: Jun. 12, 2020

(30) Foreign Application Priority Data
Dec. 15, 2017 (DE) 10 2017 130 107.0

(51) Int. Cl. A61B 5/01; A61B 5/00; A61M 1/36; A61M 1/16

(52) U.S. Cl. CPC A61B 5/015; A61B 5/0077; A61M 1/3655; A61M 2205/3306; A61B 5/6866; A61M 2205/3334; A61M 2230/50; A61M 1/16

(57) ABSTRACT

The present invention relates to a method for detecting the body temperature of a patient, wherein the patient has a fistula for draining blood into an extracorporeal circuit and/or for supplying blood from an extracorporeal circuit, wherein a thermal image recording of the fistula or of a part of the fistula is produced by means of an infrared camera for the purpose of detecting the body temperature of the patient. The present invention further relates to a dialysis machine.

The present invention relates to a method and to an apparatus for detecting the body temperature, preferably the core temperature, of the body of a patient, wherein the patient has a fistula for draining blood into an extracorporeal circuit and/or for supplying blood from an extracorporeal circuit.

As part of dialysis treatment, a patient monitoring is of great importance to ensure a reliable treatment course and to be able to intervene quickly and in a targeted manner on the occurrence of unwanted situations or conditions of the patient. An important patient parameter that is to be monitored during the treatment is the body temperature of the patient.

The measurement of the body temperature of the patient, for example, enables the recognition of infections, but is also of great importance as part of the heat regulation of the patient during the dialysis treatment. Hemodialysis here in particular represents a challenge for the heat regulation since the temperature of the blood approaches the temperature of the dialyzate during the treatment.

The blood is typically returned to the patient in temperature equilibrium with the dialyzate, with the heat transition from the blood to the dialyzate taking place in the dialyzer that forms a component of the extracorporeal circuit of the dialysis machine.

Depending on which temperature the dialyzate has, an increase or a decrease in the body temperature can take place, with even slight changes of the body temperature (in particular of the core temperature of the body), e.g. in the range from 0.3-0.8°, having the consequence of complex counteraction mechanisms of the body. These mechanisms serve to keep the body temperature constant in a specific range. The counteraction mechanisms or the change of the body temperature influence either the circulation stability, e.g. by expanding the vessels or by a blood pressure drop on an overheating of the blood, or the patient comfort that e.g. makes itself felt by a cold feeling, shivering, etc. on the cooling of the blood.

Against this background it is important to keep the body temperature of the patient constant or substantially constant during the treatment, i.e. to keep the patient “isothermal”. This can be achieved in that the blood temperature of the patient is monitored and in that the dialysis temperature is adapted accordingly.

The measurement of the body temperature can take place, for example, using a commercial clinical thermometer or also by means of an ear or forehead thermometer.

It is disadvantageous with this measurement method that the body temperature cannot be precisely detected depending on the site of the measurement (oral, rectal, armpit, eardrum), that a relatively long measurement time is required, and that the measurement result is influenced by the environmental conditions and can also be subject to artifacts. It is disadvantageous in another respect that manipulations have to be carried out on the patient that may be felt to be uncomfortable by him or that are unpractical for the course of the dialysis treatment in total.

A further disadvantage of these known measurement methods comprises their not being able to be carried out continuously, but rather only at time intervals, that is that only an intermittent measurement of the temperature is possible.

A continuous temperature measurement can admittedly take place in the patient's esophagus, but this requires an invasive procedure which is correspondingly unpleasant for the patient and in which a temperature sensor is introduced as a probe into the esophagus of the patient and remains there for the duration of the measurement.

The aforesaid measurement processes are above all disadvantageous for a dialysis treatment because the measurement is associated with delays and changes of the body temperature can thus only be reproduced or detected with a delay. This precludes or makes more difficult a very fast response of the device or of the personnel to unwanted temperature changes of the patient.

A fast temperature measurement of the blood is possible by means of an infrared sensor at the blood hose of the extracorporeal circuit. This method is, however, technically complex and cost-intensive.

It is the underlying object of the present invention to provide a method and an apparatus for detecting the body temperature, preferably the core body temperature, that can be used during a dialysis treatment and by means of which the body temperature or a parameter representative thereof can be determined simply and precisely.

This object is achieved by a method having the features of claim 1.

Provision is accordingly made that a thermal image recording, in the following also simply called a “thermal image”, of the fistula or of a part of the fistula is made by means of an infrared camera for the purpose of detecting the body temperature of the patient. It is thus the underlying idea of the present invention to apply a thermal image measurement by means of an infrared camera known from the prior art to the surface of the fistula or of the skin in the region of the fistula. “Detecting the body temperature” is not necessarily to be understood such that a temperature value of e.g. 37° C. is measured. The term also includes the finding of a value representative of the temperature such as the color of the thermal image in the region of the fistula.

A conclusion can be drawn on the core body temperature, on the skin temperature, on the blood temperature, or on another body temperature of the patient from the thermal image of the fistula or of the skin over the fistula created in this manner.

A fistula is preferably understood as a connection that is not natural, but is rather surgically inserted between an artery and a vein of the patient. It can consist of a natural or synthetic fabric.

The term “body temperature” is to be understood as wide and comprises the temperature at any desired point of the body such as on the surface of the skin, in the blood, etc.

The term “body temperature” is preferably to be understood as the “core body temperature”.

The recording of the thermal image preferably takes place contactlessly, i.e. without touching the patient, so that he is not impaired in any way. The recording can be carried out comparatively simply and also precisely. The different colors or color tones represent the respective temperatures present.

The measurement or the recording can take place without any great technical effort and is comfortable for the patient. Expensive components and technically particularly complex components are not required.

The recording of the thermal image can take place continuously or also at specific or irregular time intervals, e.g. on the occurrence of particular events that make a check of the body temperature appear necessary.

In a preferred embodiment of the invention, the temperature of the skin of the patient can be measured, i.e. a conclusion can be drawn on a specific temperature value, by means of the thermal image recording. The conversion or “calculation” of the thermal image to temperature values is, however, not an absolute requirement of the invention. It is generally also conceivable that the measured color in the region of the fistula is compared with a reference value or with a reference color and that on this bases a determination is made whether the temperature of the patent is in a desired range or not.

The recording or the temperature measurement preferably takes place in the infrared spectral range (IR) using an array, i.e. using a two-dimensional measurement field.

The maximum temperature over the IR surface is preferably determined. Movements of the patient's arm can also be detected and thus do not influence the measurement result due to the two-dimensional measurement and the determination of the maximum temperature.

It is advantageous that the surface temperature of the skin of the affected arm largely agrees with the internal fistula temperature so that a conclusion can be drawn from the determined skin temperature on the fistula temperature and thus on the blood temperature. The detection of the internal fistula temperature at the start of the treatment can optionally be made more precise empirically or by comparison with the skin temperature.

The internal fistula temperature determined in this manner can be equated with the core body temperature. If a cardiac output of 5 l/min is assumed, the flow in the fistula is typically 1 l/min and thus very high. This allows the conclusion that the internal fistula temperature is identical or almost (±5%) identical to the core body temperature. An algorithm for determining the core body temperature can thus be dispensed with.

The recording of the thermal image and/or the measurement of the body temperature based thereon can be carried out before and/or during and/or after a dialysis treatment.

As stated above, the thermal image recording can be made continuously or intermittently. This applies accordingly to the derivation of the body temperature on the basis of the thermal image recording.

It is furthermore conceivable that an actual value of the thermal image recording or of a parameter derived therefrom is compared with a desired value or a desired value range and that a regulation is carried out so that the actual value agrees with the desired value or is within the desired value range. A regulation of the body temperature is possible in this manner. This regulation of the body temperature can take place, for example, via a change of the throughflow and/or of the temperature of the dialyzate that flows through the dialyzer on the dialyzate side.

This regulation is preferably carried out by a regulation unit of the dialysis machine.

Other variables are also conceivable and covered by the invention.

The regulation parameter within the framework of the invention can be the temperature acquired from the thermal image or also a different parameter, for example the color of the thermal image.

To be able to give the patient or the medical staff an indication of the temperature of the patient, it is conceivable that a deviation between the actual value and the desired value or the desired value range is e.g. numerically displayed and that a corresponding display apparatus is thus e.g. provided at the dialysis machine.

The present invention furthermore relates to an apparatus in the form of a dialysis machine such as a hemodialysis machine, a hemofiltration machine, or a hemodiafiltration machine having an extracorporeal blood circuit in which a dialyzer is located that is flowed through by blood of the patient in operation of the machine, wherein the dialysis machine has a thermal image camera that is suitable and oriented to produce a thermal image recording of the fistula or a part of the fistula of the patient connected to the dialysis machine.

The thermal image camera can e.g., be located at the IV pole of the dialysis machine and/or can form an integral component of the dialysis machine.

The thermal image camera can, for example, be formed as an infrared gun or by another mobile device, which brings about the advantage that a physician can move from one treatment position to the next and can carry out thermal image recordings fast and simply.

The recording range of the thermal image camera is preferably variable so that it can, for example, track the movement of the arm of the patient and thus also the movement of the fistula.

This tracking can take place manually or also automatically, i.e. by the dialysis machine, for example in that the latter has the thermal image camera track a reference point. This reference point does not change its position relative to the fistula.

The reference point can, for example, be the warmest position of the arm in the region recorded by the thermal image camera.

The dialysis machine dialysis machine can have a regulator by means of which the temperature in the fistula or in the region of the fistula can be held at a desired value or in a desired value range. It is thus possible e.g. to maintain the core body temperature of the patient at a desired value or in a desired value range during a dialysis treatment. This regulation is preferably carried out autonomously by the dialysis machine so that no intervention of the user is required.

Provision is preferably made that the dialyzer is flowed through by dialyzate at the dialyzate side in operation of the machine and that the regulator is configured such that the temperature and/or the flow rate of the dialyzate is/are variable as control variables by the regulator. The temperature of the blood flowing through the dialyzer at the blood side can be kept constant, increased or decreased by the temperature and/or by the flow rate of the dialyzate. The dialyzer preferably comprises a membrane or a plurality of membranes, preferably hollow fiber membranes, that separate the blood side from the dialyzate side and via which a substance and thermal transfer is possible.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be explained in more detail with reference to an embodiment shown in the drawing.

There are shown:

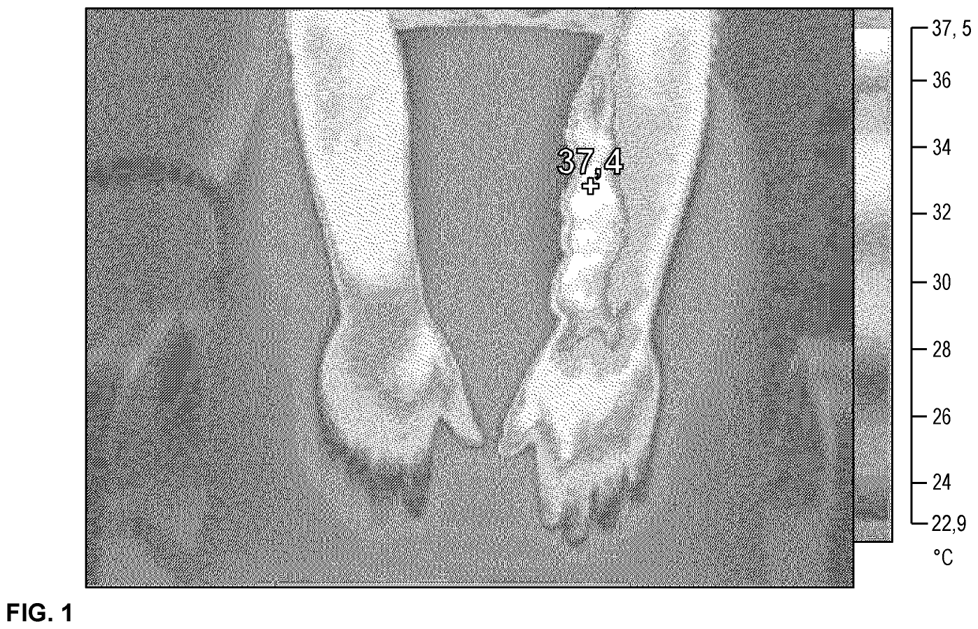

FIG. 1: a thermal image recording of second lower arms, with a fistula being arranged in the arm shown at the right; and

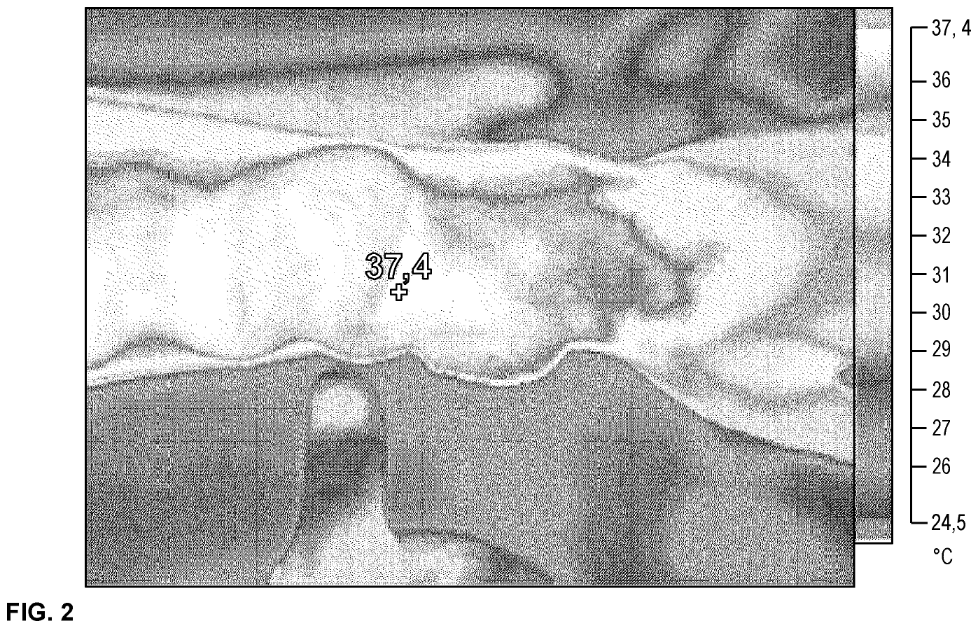

FIG. 2: a thermal image recording of the arm shown at the right in FIG. 1 with a temperature indication in the fistula.

A thermal image recording of second lower arms of a patient can be recognized from FIG. 1, with the different colors or gray shades representing different temperatures. A temperature scale is indicated at the right of FIG. 1 that enables an association of colors or gray shades to temperature values.

It can be seen from FIG. 1 that the arm shown at the right has the biggest temperature in the region marked by the cross at which the fistula is located.

This results from the enlarged representation in accordance with FIG. 2. It results from this that an actual temperature of 37.4° results for the fistula and thus ultimately also for the blood flowing through the fistula and thus also for the core body temperature.

This value is within a desired value range so that no measures have to be taken for the temperature change.

If the temperature of the blood in the fistula is, however, too low, its increase can e.g. be achieved by a heating of the dialyzate that flows through the dialyzer of the extracorporeal circuit at the dialyzate side. If the measured temperature is too high, a reduction of the blood temperature can accordingly be achieved by a reduction of the temperature of the dialyzate.

It is comparatively simple to draw possible conclusions on the core temperature of a patient through the present invention. It is possible on this basis to regulate this to a desired value range to ensure that the patient is not at risk during the dialysis treatment and feels well during the treatment. Complex and/or expensive or even invasive apparatus for measuring the body temperature are not required.

1. A method for detecting the body temperature of a patient, wherein the patient has a fistula for draining blood into an extracorporeal circuit and/or for supplying blood from an extracorporeal circuit, characterized in that a thermal image recording of the fistula or of a part of the fistula is produced by means of an infrared camera for the purpose of detecting the body temperature of the patient.

2. A method in accordance with claim 1, characterized in that the temperature of the skin of the patient is detected by means of the thermal image recording; and/or in that a conclusion is drawn from this detected temperature value of the skin on the blood temperature in the fistula and/or on the core body temperature.

3. A method in accordance with claim 1, characterized in that the body temperature is measured before and/or during and/or after a dialysis treatment.

4. A method in accordance with claim 1, characterized in that the thermal image recording is made continuously or intermittently.

5. A method in accordance with claim 1, characterized in that the measurement of the body temperature is carried out continuously or intermittently on the basis of the thermal image recording.

6. A method in accordance with claim 1, characterized in that an actual value of a parameter of the thermal image recording or an actual value of a parameter derived therefrom is compared with a desired value or a desired value range; and in that a regulation of the actual value is carried out so that the latter agrees with the desired value or lies within the desired value range.

7. A method in accordance with claim 6, characterized in that a deviation between the actual value and the desired value or desired value range is displayed.

8. A method in accordance with claim 1, characterized in that the range of the maximum or minimum temperature is identified; and in that a change of the position of this range is taken into account in the detection or evaluation of the thermal image recording.

9. A method in accordance with claim 1, characterized in that the thermal image recording is taken in a two-dimensional array.

10. A method in accordance with claim 1, characterized in that the thermal image recording is made by an infrared gun or by another mobile device.

11. A dialysis machine having an extracorporeal blood circuit in which a dialyzer is located that is flowed through by blood of a patient having a fistula in operation of the machine, characterized in that the dialysis machine has a thermal image camera that is suitable and oriented to produce a thermal image recording of the fistula or of a part of the fistula of the patient.

12. A dialysis machine in accordance with claim 11, characterized in that the thermal image camera is located at the IV pole of the dialysis machine and/or forms an integral component of the dialysis machine; and/or in that the recording range of the thermal image camera is variable.

13. A dialysis machine in accordance with claim 11, characterized in that the dialysis machine has determination means by means of which a temperature value can be determined from the thermal image recording.

14. A dialysis machine in accordance with claim 11, characterized in that the dialysis machine has a regulator by means of which the temperature of the skin over the fistula or the temperature in the fistula or in the region of the fistula can be maintained at a desired value or in a desired value range.

15. A dialysis machine in accordance with claim 14, characterized in that the dialyzer is flowed through by dialyzate at the dialyzate side in operation of the machine; and in that the regulator is configured such that the temperature and/or the flow rate of the dialyzate is/are variable as control variables by the regulator for the purpose of regulation.
